(19) United States
(12) Patent Application Publication
(10) Pub. No.: US 2005/0080288 A1
(43) Pub. Date: Apr. 14, 2005

(54) Process for the preparation of 1-[cyano(phenyl)methyl]cyclohexanol compounds

(75) Inventors: Huldreich Trafelet, Strengelbach (CH); Hans-Rudolf Marti, Oftringen (CH)

Correspondence Address: WYETH;PATENT LAW GROUP, 5 GIRALDA FARMS, MADISON, NJ, 07940, US

(73) Assignee: Wyeth, Madison, NJ (US)

(21) Appl. No.: 10/956,970

(22) Filed: Oct. 1, 2004

(30) Foreign Application Priority Data
Oct. 2, 2003 (CH) 01672/03

(51) Int. Cl. C07C 253/30

(52) U.S. Cl. 558/357

(57) ABSTRACT

Process for the preparation of 1-[cyano(phenyl)methyl]cyclohexanol compounds of general formula (I): 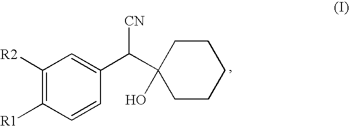 in which
R₁ is hydrogen or (C₁₋₄)alkoxy, and
R₂ is hydrogen, (C₁₋₄)alkyl or (C₁₋₄)alkoxy,
   by reacting a compound of general formula (II): 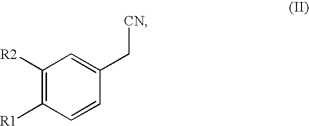
   in which R₁ and R₂ are as defined above, with cyclohexanone in the presence of a catalyst, characterized in that this catalyst is selected from the group comprising alkali metal alcoholates, alkaline earth metal alcoholates, aluminium alcoholates and tetrasubstituted ammonium hydroxide.

The present invention relates to a process for the preparation of 1-[cyano(phenyl)-methyl]cyclohexanol compounds, e.g. the compound 1-[cyano(4-methoxyphenyl)-methyl]cyclohexanol, which represents an important intermediate for the preparation of venlafaxine.

1-[Cyano(phenyl)methyl]cyclohexanol compounds are known per se, for example from EP 0 112 669. However, there is still a need to prepare these compounds with cost-effective starting materials and a high purity of the products obtained, i.e. with the minimum of expenditure on the work-up and purification of the reaction products, at higher reaction temperatures and for shorter reaction times.

The present invention relates to a process for the preparation of 1-[cyano(phenyl)-methyl]cyclohexanol compounds of general formula (I): 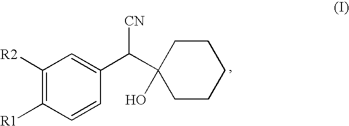
in which
R₁ is hydrogen or (C₁₋₄)alkoxy, and
R₂ is hydrogen, (C₁₋₄)alkyl or (C₁₋₄)alkoxy,
by reacting a compound of general formula (II): 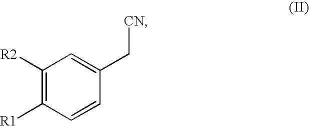
   in which R₁ and R₂ are as defined above, with cyclohexanone in the presence of a catalyst, characterized in that this catalyst is selected from the group comprising alkali metal alcoholates, alkaline earth metal alcoholates, aluminium alcoholates and tetrasubstituted ammonium hydroxides, preferably alkali metal and/or alkaline earth metal alcoholates and tetrasubstituted ammonium hydroxides. The present invention further relates to the compounds prepared by this process.

The reaction can be carried out in the presence of a suitable inert solvent or without the addition of a solvent. Examples of suitable solvents are pentane, hexane, heptane, benzene, toluene, diethyl ether or related solvents. The choice of solvents is familiar to those skilled in the art. The reaction is preferably carried out without the addition of a solvent.

R₁ is preferably (C₁₋₄)alkoxy, particularly preferably methoxy or ethoxy and very particularly preferably methoxy.

R₂ is preferably hydrogen or methyl and particularly preferably hydrogen.

Preferably, the compound 1-[cyano(4-methoxyphenyl)methyl]cyclohexanol is prepared according to the invention.

Examples of preferred catalysts from the group comprising alkali metal alcoholates are the sodium and potassium alcoholates known per se, especially the sodium and potassium alcoholates of methanol, ethanol, n-propanol, sec-propanol, n-butanol, sec-butanol and tert-butanol. The sodium and potassium alcoholates of ethanol and tert-butanol are preferred and potassium tert-butylate is particularly preferred.

Preferred catalysts from the group comprising alkaline earth metal alcoholates are the magnesium alcoholates known per se, especially the magnesium alcoholates of methanol, ethanol, n-propanol, sec-propanol, n-butanol, sec-butanol and tert-butanol, the magnesium alcoholates of ethanol and tert-butanol being particularly preferred and magnesium tert-butylate being very particularly preferred.

The aluminium alcoholate catalysts used are preferably the aluminium alcoholates of methanol, ethanol, n-propanol, sec-propanol, n-butanol, sec-butanol and tert-butanol, the aluminium alcoholates of ethanol and tert-butanol being particularly preferred and aluminium tert-butylate being very particularly preferred.

Examples of preferred catalysts from the group comprising tetrasubstituted ammonium hydroxides are tetra(C₁₋₄)alkylammonium hydroxides such as tetrabutylammonium hydroxide, and tri(C₁₋₄)alkyl(benzyl)ammonium hydroxides such as triethyl(benzyl)ammonium hydroxide. Tetrabutylammonium hydroxide is particularly preferred.

The amount of catalyst in the reaction mixture is in the range from about 0.1 to 1.0 mol %, preferably between 0.1 and 0.3 mol % and particularly preferably about 0.2 mol % of catalyst per mol of compound of formula (II).

The reaction is carried out by mixing the two starting materials, i.e. cyclohexanone and the compound of formula (II), and the catalyst, in any order, at a temperature below 30° C. (<30° C.), at which point the reaction starts. Preferably, the compound of formula (II) is mixed with the catalyst and then the cyclohexanone is added. The preferred reaction temperature is in the range from 15° C. to 25° C. It is preferable to use an excess of cyclohexanone, preferably a 20-60 mol % excess of cyclohexanone, based on the compound of formula (II). However, the reaction can equally well be carried out with molar amounts. The reaction time ranges from about 10 minutes to 24 hours and preferably from about 15 minutes to 120 minutes. The product can then be isolated, after the addition of solvent if necessary, and optionally purified further in a manner known per se. The Examples which follow illustrate the invention.

### EXAMPLE 1

A mixture containing 1.0 eq. of 4-methoxybenzyl cyanide, 1.4 eq. of cyclohexanone and 0.2 mol % of tetrabutylammonium hydroxide hydrate is stirred for 15 minutes at room temperature. The exothermicity is absorbed by cooling to keep the reaction temperature at about 20° C. to 25° C. This gives a thick white mass to which toluene and a little dilute aqueous hydrochloric acid (0.1 molar) are added, the reaction product dissolving in the toluene. The mixture is heated to about 30° C. and the organic phase is separated off and washed with pure water. The organic phase is concentrated and heptane is added. The solution is then cooled to about 0° C. and stirred for a further 30 minutes. The crystalline 1-[cyano(4-methoxyphenyl)methyl]cyclohexanol is isolated in a purity of over 98% and in a yield of 73.6 mol % of crystalline material, based on the 4-methoxybenzyl cyanide used.

### EXAMPLE 2

A mixture containing 1 eq. of 4-methoxybenzyl cyanide, 1.4 eq. of cyclohexanone and 0.2 mol % of potassium tert-butylate is stirred for about 30 minutes at room temperature. The exothermicity is absorbed by cooling to keep the reaction temperature in the range from 20° C. to 25° C. The thick white suspension is diluted with heptane and adjusted to pH 3-4 with a little acetic acid. The suspension is then cooled to a temperature below 10° C. (<10° C.) and stirred for a further 30 minutes. The crystalline product is filtered off and washed with a little heptane to give 1-[cyano(4-methoxyphenyl)methyl]cyclohexanol in a purity of 98.4% and in a yield of 82.4 mol % of crystalline material, based on the 4-methoxybenzyl cyanide used.

### EXAMPLE 3

0.2 mol % of potassium tert-butylate is added at room temperature to a mixture consisting of 1.0 eq. of 4-methoxybenzyl cyanide and 1.4 eq. of cyclohexanone in 0.71 part of toluene (based on 4-methoxybenzyl cyanide) and the reaction mixture is stirred for 24 hours at this temperature, during which time it warms up to 26° C. The reaction mixture is then adjusted to pH 3-4 with a little acetic acid, diluted with heptane, adjusted to a temperature below 10° C. (<10° C.) and stirred for a further 30 minutes. The resulting product is then filtered off to give 1-[cyano(4-methoxy-phenyl)methyl]cyclohexanol in a purity of 98.6% and in a yield of 68.2 mol % of crystalline material, based on the 4-methoxybenzyl cyanide used.

### EXAMPLE 4

0.2 mol % of potassium tert-butylate is added to a mixture of 1.0 eq. of 4-methoxy-benzyl cyanide and 1.4 eq. of cyclohexanone in 1.3 parts of heptane (based on 4-methoxybenzyl cyanide) and the resulting mixture is stirred for 50 minutes at room temperature. The exothermicity is absorbed by cooling. The procedure is then continued as described in Example 3 to give 1-[cyano(4-methoxyphenyl)-methyl]cyclohexanol in a purity of 98.6% and in a yield of 88.8 mol % of crystalline material, based on the 4-methoxybenzyl cyanide used.

1. Process for the preparation of 1-[cyano(phenyl)methyl]cyclohexanol compounds of general formula (I): 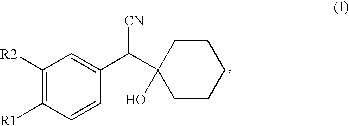
in which
R₁ is hydrogen or (C₁₋₄)alkoxy, and
R₂ is hydrogen, (C₁₋₄)alkyl or (C₁₋₄)alkoxy,
comprising reacting a compound of general formula (II): 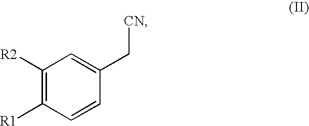
in which R₁ and R₂ are as defined above, with cyclohexanone in the presence of a catalyst selected from alkali metal alcoholates, alkaline earth metal alcoholates, aluminium alcoholates and tetrasubstituted ammonium hydroxides

2. The process of claim 1, wherein the reaction is carried out in the presence of a suitable inert solvent.

3. The process of claim 1, wherein the reaction is carried out without the addition of a solvent.

4. The process of claim 1 wherein R₁ is (C₁₋₄)alkoxy.

5. The process of claim 4 wherein R₁ is methoxy.

6. The process of claim 1 wherein R₂ is hydrogen or methyl.

7. The process of claim 6 wherein R₂ is hydrogen.

8. The process of claim 1 wherein the compound of Formula I is 1-[cyano(4-methoxyphenyl)methyl]cyclohexanol.

9. The process of claim 1 wherein the catalyst is an alkali metal alcoholate.

10. The process of claim 9 wherein the alkali metal alcoholate catalyst is sodium or potassium alcoholate.

11. The process of claim 10 wherein the alkali metal alcoholate catalyst is a sodium or potassium alcoholate of methanol, ethanol, n-propanol, sec-propanol, n-butanol, sec-butanol or tert-butanol.

12. The process of claim 11 wherein the catalyst is potassium tert-butylate.

13. The process of claim 1 wherein the catalyst is an alkaline earth metal alcoholate.

14. The process of claim 13 wherein the alkaline earth metal alcoholate catalyst is a magnesium alcoholate.

15. The process of claim 14 wherein the alkaline earth metal alcoholate catalyst is a magnesium alcoholate of methanol, ethanol, n-propanol, sec-propanol, n-butanol, sec-butanol or tert-butanol.

16. The process of claim 15 wherein the catalyst is magnesium tert-butylate.

17. The process of claim 1 wherein the catalyst is an aluminium alcoholate.

18. The process of claim 17 wherein the catalyst is an aluminium alcoholate of methanol, ethanol, n-propanol, sec-propanol, n-butanol, sec-butanol or tert-butanol.

19. The process of claim 18 wherein the catalyst is aluminium tert-butylate.

20. The process of claim 1 wherein the catalyst is a tetra(C₁₋₄)alkylammonium hydroxide.

21. The process of claim 20 wherein the tetra(C₁₋₄)alkylammonium hydroxide catalyst is tetrabutylammonium hydroxide, or a tri(C₁₋₄)alkyl(benzyl)ammonium hydroxide.

22. The process of claim 21 wherein the catalyst is triethyl(benzyl)ammonium hydroxide.

23. The process of claim 1 wherein the catalyst is used in the reaction mixture in an amount of about 0.1 to about 1.0 mol % per mol of compound of Formula II.

24. The process of claim 23 wherein the catalyst mol % of catalyst is from about 0.1 to about 0.3 mol % of catalyst per mol of compound of formula (II).

25. The process of claim 24 wherein the catalyst mol % of catalyst is about 0.2 mol % of catalyst per mol of compound of formula (II).

26. The process of claim 1 wherein the reaction is performed at less than about 30° C.

27. The process of claim 1 wherein the reaction is performed at from about 15° C. to about 25° C.

28. The process of claim 1 wherein compound of formula II is mixed with the catalyst before the addition of cyclohexanone.

29. The process of claim 1 wherein cyclohexanone is used in at least about 20 to about 60 mol % excess based on the compound of formula (II).

30. Compound prepared according the process of claim 1.
